(19) United States
(12) Patent Application Publication
(10) Pub. No.: US 2024/0341842 A1
(43) Pub. Date: Oct. 17, 2024

(54) DEVICES, SYSTEMS, AND METHODS FOR USE DURING TRANSCATHETER AORTIC VALVE REPLACEMENT

(71) Applicant: Kar Health, LLC, Iowa City, IA (US)

(72) Inventors: Wassef Karrowni, Cedar Rapids, IA (US); Mirna Karouni, Cedar Rapids, IA (US)

(21) Appl. No.: 18/598,936

(22) Filed: Mar. 7, 2024

Related U.S. Application Data
(60) Provisional application No. 63/450,426, filed on Mar. 7, 2023.

(51) Int. Cl. A61B 18/14; A61B 18/00; A61B 18/02; A61B 18/04; A61B 18/18; A61B 18/24

(52) U.S. Cl. CPC A61B 18/1492; A61B 18/02; A61B 18/1815; A61B 18/24; A61B 2018/00184; A61B 2018/0022; A61B 2018/00267; A61B 2018/00369; A61B 2018/00577; A61B 2018/00601; A61B 2018/00607; A61B 2018/00702; A61B 2018/00714; A61B 2018/00732; A61B 2018/00761; A61B 2018/0212; A61B 2018/046; A61B 2018/142; A61B 2018/1435; A61B 2018/1475; A61B 2018/1861

(57) ABSTRACT

Catheters and functional aspects thereof for use during transcatheter aortic valve replacement (TAVR), and for prevention of coronary obstruction and/or sinus sequestration. A method valve leaflet modification comprising inserting a catheter into a vessel to a target valve, positioning a distal end of the catheter adjacent to a leaflet of the target valve, and lacerating the leaflet with a functional aspect of the catheter.

### CROSS-REFERENCE TO RELATED APPLICATION(S)

This application claims the benefit under 35 U.S.C. § 119 (e) to U.S. Provisional Application 63/450,426, filed Mar. 7, 2023, and entitled Devices, Systems, and Methods for Use During Transcatheter Aortic Valve Replacement, which is hereby incorporated herein by reference in its entirety for all purposes.

### TECHNICAL FIELD

The disclosure relates to devices, systems, and methods for use during transcatheter aortic valve replacement (TAVR), and more particularly relate to prevention of coronary obstruction and/or sinus sequestration.

### BACKGROUND

Transcatheter aortic valve replacement (TAVR) is an effective and minimally invasive procedure to treat aortic valve stenosis. Typically, a computed tomography (CT) scan is performed in preparation for TAVR in order to study the anatomy of the existing aortic valve. Results from a CT scan can be used to decide on the size of the TAVR bioprosthetic valve that needs to be implanted, and also to evaluate for potential procedure related complications. Some of the anatomical variables to study include the distance from the origin of the coronary arteries to the aortic valve annulus, the length and bulkiness of valve leaflets, the height of the sinotubular junction, and others as would be appreciated. Studying these parameters is of critical importance to avoid a known complication of TAVR which is occlusion of the coronary artery, which can occur when the scaffold of the new valve pushes the existing leaflets into the coronary arteries.

As would be appreciated by those in the art, TAVR is expected to be used more frequently in the future in cases where a patient has a failed existing bioprosthetic valve (whether a prior surgical valve or TAVR valve). In these cases of valve-in-valve (VIV) TAVR, there is increased risk of occlusion of one or both coronary ostia and risk of sequestration or effacement of the sinuses of Valsalva. This is because the implanted TAVR valve, pins open the leaflets of the first existing prosthesis. Depending on the anatomy of the patient this type of procedure might create a tube graft or covered stent within the ascending aorta, which sequesters the sinuses of Valsalva and obstructs blood flow to the coronary arteries.

BASILICA (Bioprosthetic or Native Aortic Scallop Intentional Laceration to Prevent Iatrogenic Coronary Artery Obstruction), is a technique currently being used in clinical practice to prevent coronary obstruction following TAVR. As would be understood, BASILICA uses transcatheter electrosurgery to lacerate or split the aortic leaflets before TAVR allowing the leaflets to splay to the sides after TAVR and, thus allows for flow of blood through the laceration into the coronary sinus and coronary artery ostium. The impact of BASILICA on flow patterns and washout of the coronary sinuses is currently being studied. The BASILICA technique has several limitations including that it is technically challenging and requires a high level of expertise, it might not be effective in calcified leaflets that prohibit traversal by a wire, and it might not be suitable in valve-in-valve cases especially in TAVR-in-TAVR cases because of transcatheter valve design and because of randomness of commissural alignment.

There is a need in the art for improved devices, systems, and methods for preventing complications during and following TAVR.

### BRIEF SUMMARY

Disclosed herein are various techniques and related devices and systems for minimally invasive and catheter-based materials and methods to modify a native or bioprosthetic aortic valve leaflet by ablation in order to lacerate, fenestrate, or retract one or more leaflets to prevent coronary obstruction and/or sinus sequestration during TAVR. In various implementations, this is achieved using a catheter with an expandable device or a pre-shaped catheter to deliver targeted and controlled modification or ablation to the leaflets of the native or bioprosthetic aortic valve. In various implementations, the catheter or segments thereof deliver energy from an energy source to aortic valve leaflet tissue in a directed manner in order to avoid damage to adjacent tissue.

In Example 1, a catheter comprising an elongate shaft comprising proximal and distal ends, a lumen, and a functional aspect configured to navigate to and ablate aortic valve leaflet tissue inside a subject sinus of Valsalva.

Example 2 relates to the catheter of any of Examples 1 and 3-13, wherein the functional aspect is an inflatable balloon or expandable member.

Example 3 relates to the catheter of any of Examples 1-2 and 4-13, wherein the functional aspect is configured for targeted delivery of ablation energy or modification therapy to aortic valve leaflets.

Example 4 relates to the catheter of any of Examples 1-3 and 5-13, wherein the functional aspect is configured for directional or differential delivery of ablation energy via electrodes to a leaflet of the aortic valve inside the sinus of Valsalva.

Example 5 relates to the catheter of any of Examples 1-4 and 6-13, wherein the functional aspect comprises an active surface facing the valve leaflet tissue and passive or inert surface facing the aortic root wall in native valves or the cage of the bioprosthesis.

Example 6 relates to the catheter of any of Examples 1-5 and 7-13, wherein the functional aspect is circular, semilunar or oval shaped.

Example 7 relates to the catheter of any of Examples 1-6 and 8-13, further comprising at least one electrode wherein the electrode disposed on a surface of the expandable member or embodied in the functional aspect.

Example 8 relates to the catheter of any of Examples 1-7 and 9-13, wherein the functional aspect is configured to apply energy to a leaflet of the aortic valve inside the sinus of Valsalva, the energy selected from the group consisting of electrical, thermal, radiofrequency (RFA), laser, microwave, ultrasound, ultraviolet light, pulse field or cooling energy.

Example 9 relates to the catheter of any of Examples 1-8 and 10-13, configured to control at least one of frequency, duration, temperature, and power of the energy application.

Example 10 relates to the catheter of any of Examples 1-9 and 11-13, wherein the functional aspect is a pigtail-shaped extension comprising one or more electrodes.

Example 11 relates to the catheter of any of Examples 1-10 and 12-13, wherein the functional aspect is a clamp comprising first and second arms configured to grasp the valve leaflet and deliver focused ablation energy between the first and second arms.

Example 12 relates to the catheter of any of Examples 1-11 and 13, wherein the energy delivered by the clamp can be used to coagulate grasped leaflet tissue and/or cut the grasped leaflet tissue.

Example 13 relates to the catheter of any of Examples 1-13, further comprising a cutting device disposed between the first and second arms.

In Example 14 a method for performing transcatheter aortic valve replacement comprising inserting a catheter into an artery to a target valve, positioning a distal end of the catheter adjacent to a leaflet of the target valve, and lacerating the leaflet with a functional aspect of the catheter.

Example 15 relates to the method of any of Examples 14 and 16-19, wherein the functional aspect is a clamp.

Example 16 relates to the method of any of Examples 14-15 and 17-19, wherein the functional aspect is an expandable member having an active surface for transferring energy for lacerating the leaflet.

Example 17 relates to the method of any of Examples 14-16 and 18-19, wherein the expandable member is a balloon.

Example 18 relates to the method of any of Examples 14-17 and 19, wherein the active surfaces comprises one or more electrodes.

Example 19 relates to the method of any of Example 14-18, wherein the functional aspect of the catheter is a balloon and further comprising expanding the balloon via a heatable fluid, wherein the heatable fluid lacerates the leaflet.

In Example 20, a method of valvular repair comprising placing a catheter at a target valve, expanding an expandable member to contact a leaflet of the target valve, the expandable member at a distal end of the catheter, and lacerating the leaflet via radiofrequency energy transfer to one or more electrodes on a surface of the expandable member.

While multiple implementations are disclosed, still other implementations of the disclosure will become apparent to those skilled in the art from the following detailed description, which shows and describes illustrative implementations of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a schematic depiction of the a catheter with a balloon in an expanded configuration within the sinus of Valsalva of the aortic valve and in contact with an aortic valve leaflet, according to one implementation.

FIG. 2 is a schematic depiction of a catheter with a metallic ball in an expanded configuration within the sinus of Valsalva of the aortic root in contact with an aortic valve leaflet, according to one implementation.

FIG. 3 is a schematic depiction of a catheter with a pigtail extension at the distal end of the catheter, according to one implementation.

FIG. 4 is a schematic depiction of the catheter with an end-to-end lumen for wire advancement, a lumen for balloon inflation, a lumen for pressure transducing, and an insulated lumen for cable energy transfer from generator to expandable member, according to one implementation.

FIG. 5 is a schematic depiction of a catheter having a clamp for grasping a valve leaflet and deliver focused ablation energy, according to one implementation.

FIG. 6 is a schematic depiction of a catheter having parallel electrodes about the balloon, according to one implementation.

FIG. 7 is a schematic depiction of the catheter having electrodes disposed equatorially about the balloon, according to one implementation.

FIG. 8 is a schematic depiction of the catheter having an electrode or other energy transducer positioned in the center of the expandable member rather than the surface, according to one implementation.

### DETAILED DESCRIPTION

Described herein are catheters and functional aspects thereof for use during transcatheter aortic valve replacement (TAVR), and for the prevention of coronary obstruction and/or sinus sequestration. Also described herein are methods valve leaflet modification comprising inserting a catheter into a vessel to a target valve, positioning a distal end of the catheter adjacent to a leaflet of the target valve, and lacerating the leaflet with a functional aspect of the catheter.

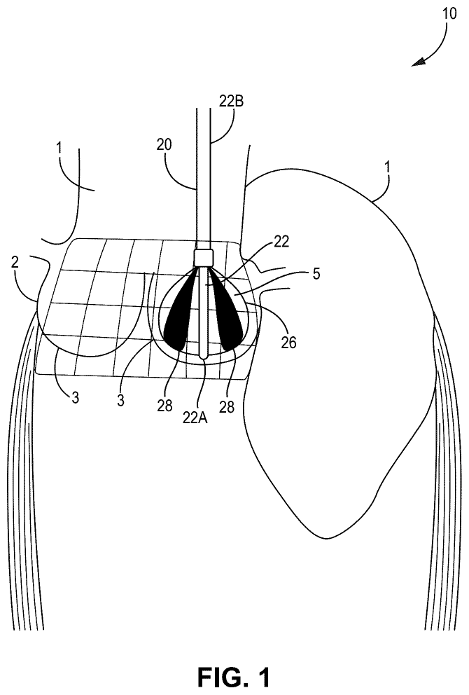

Various implementations of the disclosed system 10 include a catheter 20, as is shown generally in FIG. 1. The catheter 20, according to various implementations, includes an elongated flexible shaft 22 having distal 22A and proximal 22B ends, as would be understood. The catheter being configured for insertion into the body of a patient via known techniques.

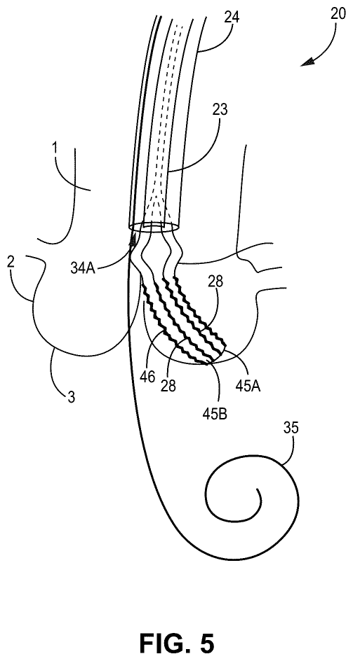

In various implementations, the catheter 20 can be inserted into the body via an artery through a standard arterial sheath or other sheath 24 (such as that shown in FIG. 5) and as will be discussed further herein. Various procedures, methods, and devices for insertion and placement of a catheter are will known in the art.

In various implementations, the catheter 20 shaft 22 is made from a flexible material or materials, such as woven Dacron or synthetic materials such as polyurethane or others, so as to be positionable and steerable. That is the length of the catheter 20 (the shaft 22) is made from material(s) that allow the catheter 20 to be positioned and optionally steered into place within a patient, as would be understood generally by those of skill in the art.

Further, as shown for example in FIG. 1, the catheter shaft 22 according to certain implementations comprises a functional aspect 5 which can be an inflatable balloon 26 or other expandable member 26 (as shown in further implementations at 40) at the distal end 22A of the catheter 20. In various implementations, the balloon 26 can be compliant, semi-complaint, or non-compliant. In certain implementations, the balloon 26 can be filled with contrast, saline, or other fluid mixture depending on the procedure and/or type of energy to be delivered. It is appreciated that the size of the balloon 26 can vary per patient anatomy and is further appreciated that common expected sizes can range from about 3 mm to about 10 mm.

The balloon 26/functional aspect 5 may optionally be made of electroconductive elastomer such as a mixture of elastomer, polymer, and electroconductive particles, in various implementations. In various further implementations, the balloon 26 can be made of latex, silicon, polyethylene, polyurethane, polyvinyl chloride, or other material understood by those of skill in the art. The balloon 26/functional aspect 5 may be made of a variety/combination of materials, as would be appreciated.

Inflation and deflation of the balloon 26, according to certain implementations, is controlled by tubing using standard techniques. In certain implementations, the balloon 26 tipped catheter 20 can be floated inside a cardiac structure or vasculature with the balloon 26 inflated or partially inflated to avoid trauma, as would be understood by those of skill in the art.

In use according to certain implementations, the catheter 20 is advanced through a sheath 24 inserted in an artery (commonly the common femoral artery) and advanced to the ascending aorta. This advancement can also be done over a guide wire (shown at 35 in FIG. 5), using standard technique, that fits inside the lumen 34A of the catheter 20. The advancement can also be done by inflation the balloon 26 or the other expandable member once the distal end 22A of the catheter 20 is inside an artery and floating the catheter 20 to the intended location. The catheter 20 advancement can also be facilitated using guiding sheath 24 positioned in the ascending aorta. That is, placement and navigation of the catheter 20 can be done by any appreciated and appropriate technique as would be recognized by those of skill in the art.

In various implementations, the intended treatment location is the aortic valve while other treatment locations and valves are possible. The various implementations herein will be described with reference to the an aortic valve procedure for conciseness and clarity although alternatives are possible and contemplated herein. Those of skill in the art would be capable of applying the devices and techniques to alternative locations.

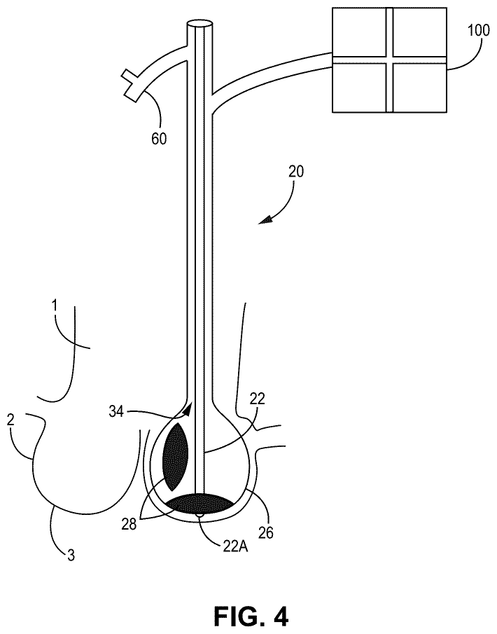

Turning to FIG. 4, once at the level of the ascending aorta/aortic root 1, the optionally balloon 26 tipped catheter 20 is advanced further (optionally with the balloon 26 inflated) into the targeted sinus of Valsalva 2 area of the aortic valve (most commonly the left coronary sinus although other locations are possible). It is appreciated that in these implementations the intended area is on the surface of the aortic valve facing the aortic root 1.

In various implementations, the advancement of the catheter 20 can be guided by fluoroscopy, echocardiographic cardiac imaging, or the like, as would be understood by those of skill in the art. Further, advancement can be facilitated by manipulation of the catheter 20 by rotation and/or pull and push movements, as would be readily appreciated. As discussed above, in various implementations, the catheter 20 is steerable.

The distal end 22A of the catheter 20 is positioned and distended to a sufficient size to ensure close contact with the aortic valve leaflet 3 inside the sinus of Valsalva 2 of the intended leaflet 3. That is the distal end 22A of the catheter 20 is disposed adjacent to the target leaflet 3. The balloon 26 or expandable member of the catheter 20 is positioned to sit in one of the sinuses of Valsalva 2 of the aortic valve in close approximation and in contact with the leaflet 3 of that sinus 2 ensuring continuous contact between the balloon 26 or expandable member and the leaflet 3. The approximation/location of the expandable member 26 against the leaflet 3 may limit the mobility of the leaflet 3. The balloon 26 or expandable member can also be used as a vehicle to deliver ablation energy to the leaflet 3 of the aortic valve while avoiding the aortic root 1 wall, as described herein.

In use according to certain implementations, once the catheter 20 is in the intended position and the balloon 26 is inflated, forward tension is applied at the catheter 20 to ensure contact of the balloon 26 or expandable member with the aortic valve leaflet 3 tissue.

After being placed, various implementations of the catheter 20 is configured to transfer energy to modify a native or bioprosthetic aortic valve leaflet 3 by ablation in order to lacerate, fenestrate, or retract one or more leaflets 3 to prevent coronary obstruction and/or sinus 2 sequestration during TAVR.

Certain implementations utilize transferred radiant energy, which according to certain implementations can be electrical, thermal, radiofrequency (RFA), laser, microwave, ultrasound, ultraviolet light, pulse field, cooling, or other energy types that would be appreciated by those of skill in the art. For example, the active surface of the balloon 26 can be used to deliver RFA by placing electrodes 28 on its surface, such as at the tip of the balloon 26 and/or equatorials. In these implementations, for example, one or more electrodes 28 can be wrapped on the exterior of the functional aspect 5, such as on the surface of the balloon 26 or on the splines of the expandable member 40. Various configurations and examples of electrodes 28 and electrode placement on the catheter 20 and functional aspect 5 are shown throughout the figures and are discussed herein.

In one implementation, the shaft 22 of the catheter 20 includes a coaxial cable insulated by a layer and transmits radiant energy from a conventional energy source 100 outside of the body to the distal end of the catheter, as shown for example in FIG. 4.

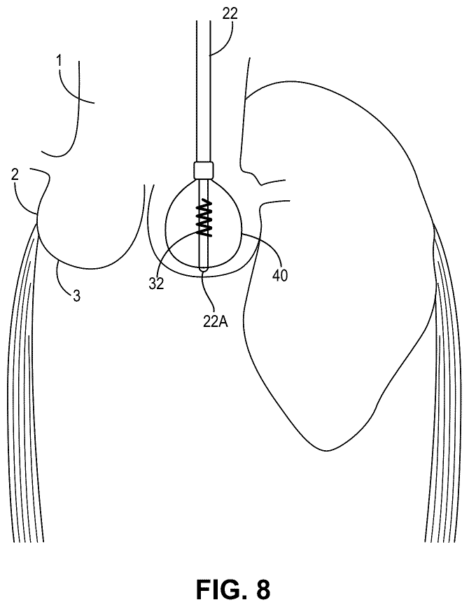

In a further implementation, the transmitted energy is transferred to a probe carrying an electrode 28 located in the shaft 22 which is an extension of the catheter 20 in the middle expandable distal end 22A, shown for example in FIG. 8.

In one application, the transmitted energy is transferred to the active surface of the expandable member 40. In another implementation, heatable fluid can be used as the medium for energy ablation. In a further implementation, thermal therapy can be delivered via an electrode 28 that is positioned in the central shaft of the balloon 26. In these and other implementations, a radiofrequency current is delivered between a coil electrode 28 mounted inside the balloon 26 and cutaneous patches, as seen for example in FIG. 8.

The amount of energy and time over which energy is delivered to the tissue can be regulated and controlled, as would be readily appreciated.

Optionally, temperature or contact sensors can be located on the surface of the expandable member 40. In further implementations, an electroconductive member is optionally disposed on the outer surface of the balloon 26.

In a still further implementation, an optical fiber with a laser energy reflector can optionally be positioned within the distal end and in the center of the catheter 20, all of which is housed in the shaft inside the inflatable balloon 26 or expandable member 40.

In certain implementations, the balloon 26 can be filled with normal saline, 5% dextrose in water, H—NaCl (for thermal energy), deuterium oxide (D2O) (for laser energy), or other fluid, as dictated by the further aspects of the implementation, as would be readily appreciated.

The shape of the balloon 26 or expandable member 40 can be round, oval, semilunar, or others as would be understood.

The balloon 26 or expandable member 40 surface can be modified to direct the application of ablation energy to one side. For example, the active surface of the balloon 26 or expandable member 40 for energy delivery may optionally be oriented towards the base of the leaflet 3 and the free-edge of the leaflet 3. In these and other implementations, the inert surface of the balloon 26 or expandable member 40 is the side(s) of the balloon 26 or expandable member 40 facing the aortic wall or the grooves of the sinuses of Valsalva 2 and/or facing the ring or cage/stent struts or prior bioprosthesis in cases of VIV, as would be understood by those of skill in the art in light of this disclosure.

As mentioned above, another implementation comprises using a distending or expandable member 40 other than balloon 26 at the tip of the catheter 20 in order to safely navigate the catheter 20 to the intended location in the sinus of Valsalva of the aortic valve, and can include using the surface of a distending or expandable member 40 at the distal tip 22A of the catheter 20 in order to deliver ablation energy.

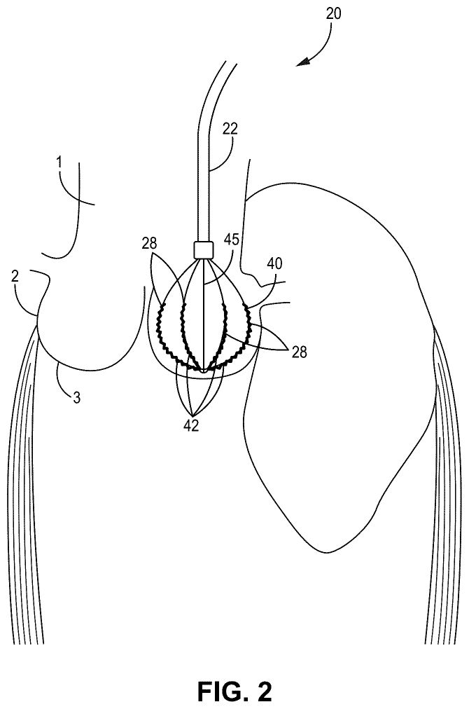

In various implementations, and as shown in FIG. 2, the distending or expandable member 40 can be lattice or ball of metal. For example, the expandable member 40 can comprise a material such as but not limited to Nitinol that can have a number of splines 42 for example five or more. Sliding the central rod 45 inside the catheter 20 proximally may form the final shape of the expandable member 40 as a ball, as would be understood. As such, sliding the central rod 45 inside the catheter 20 distally may retract the expandable member into a collapsed rod-like shape, as would be generally understood. In certain of these implementations, splines 42 of the expandable member 40 are wrapped with conductive wiring, such as an electrode 28, for conveying thermal energy.

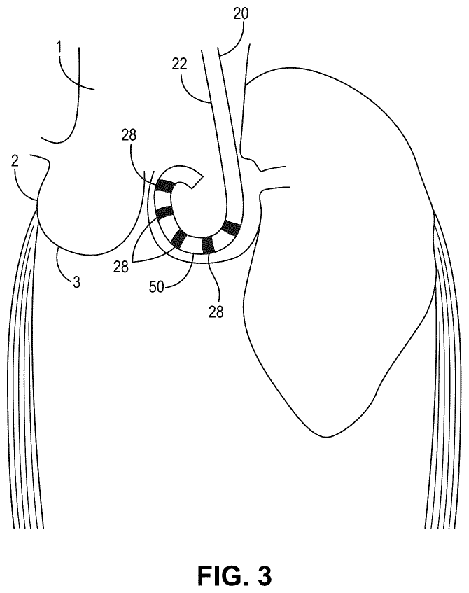

In another implementation of the catheter 20 shown in FIG. 3, the functional aspect 4 is a pigtail-shaped extension 50 of the catheter 20. In these implementations, the pigtail-shaped extension 50 is flexible and can be extended straight inside a catheter 20 or sheath 24 but once released would take its memory shape of pigtail, as is shown in FIG. 3.

It is understood, in various implementations, that the pigtail-shaped extension 50 is configured to provide safety for navigation of the catheter 20 inside the vasculature or cardiac chambers and also to facilitate positioning the pigtail end 50 of the catheter 20 in a specific coronary sinus of the aortic root 1, using standard practice techniques. The pigtail-shaped extension 50 also provides ability to control directionality of the catheter 20. For example, the catheter 20 can be rotated to position the base of the pigtail 50 in the aortic root 1 inside a sinus of Valsalva 2 facing the wall of the aorta while the free edge faces the leaflet 3. The catheter 20 can be advanced to ensure contact with the leaflet 3 tissue.

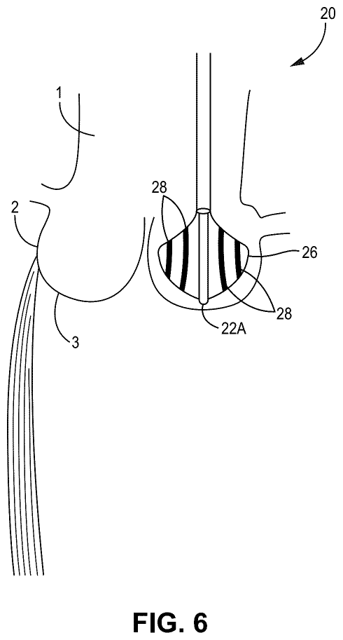

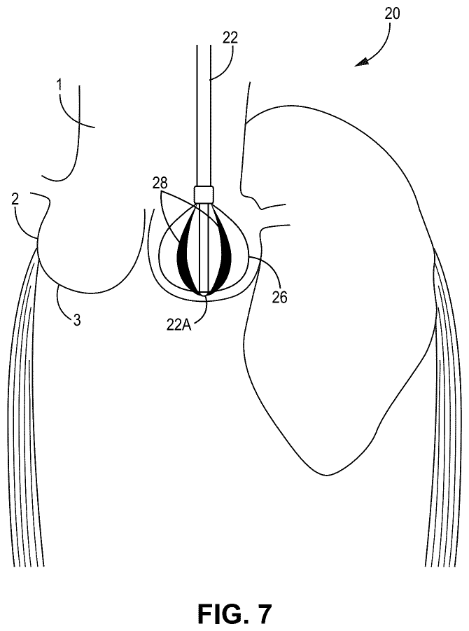

As would be appreciated, one or more electrodes 28 can be disposed about the outer surface of the pigtail-shaped extension 50 to deliver ablation energy to leaflet 3 tissue. In various implementations, these electrodes 28 are electrode rings 28. It is readily appreciated that the catheter 20 according to any of these implementations can have a varied number of electrodes 28 and electrode spacing to provide a range of options, depending on the use case. FIGS. 6-8 depict further arrangements/orientations of electrodes 28 and other components.

The electrodes 28 according to any of the implementations can be configured such that time, power, and impedance are monitored during ablation. Electrodes 28 can be made of copper, gold, graphite, titanium, brass, silver, platinum, or other understood material. Radiofrequency energy can be delivered in unipolar, bipolar, or multipolar fashion. It is further appreciated that irrigation holes can be added in the catheter 20 or on the balloon 26 or expandable member 40 to provide saline based cooling of the electrodes 28.

As shown in FIG. 4, an energy generator 100 can be in electronic communication with the electrodes 28 and be configured to control power, temperature, and duration of the applied ablation, while also measuring impedance and temperature during the procedure. FIG. 4 further depicts a port 60 in fluidic communication with the balloon 26 which is configured to facilitate inflation/deflation via, for example, the proximal lumen 34.

As shown in FIG. 5, in another implementation, the energy delivering vehicles to the leaflet 3 at the end of the catheter 20 are electrodes 28 on the surface of a clamp 46 that moves between the open position and closed position. In various implementations, the clamp 46 is made of metal (for example nitinol) that has a memory curved shape that it assumes once released from the delivery sheath 24, as would be appreciated. In various implementations, the opening and closing the jaws 46 of the catheter/device 20 can be achieved in a passive or an active way.

In one exemplary implementation, the open position is inactive relative to energy delivery and is used in order to grasp the leaflet 3 tissue between the arms 45A, 45B of the clamp 46. In the closed position, the clamp 46 becomes the active where radiofrequency energy can be delivered to the leaflet 3 tissue pinned in between the arms 45A, 45B of the clamp 46 via the electrodes 28. In certain implementations, the energy is delivered through bipolar electrodes 28 on the inner surface of both arms of the clamp 46, and can be delivered to coagulate the leaflet 3 tissue, cut the leaflet 3 tissue, or combination of both.

In various implementations, cutting of the middle of the grasped leaflet 3 tissue can also be achieved by a cutting device 23 embedded in the distal end of the catheter 20 in between and perpendicular to the first and second arms 45A, 45B of the clamp 46. The cutting part can be activated after coagulation of the grasped leaflet 3 tissue by the clamp part of the device/catheter 20.

While the leaflet 3 tissue is still grasped by the clamp 46 after coagulation, sliding the cutting device 23 forward induces cutting the leaflet 3 tissue grasped by the clamp 46. Cutting can be achieved by mechanical force (sharp edge of the cutting catheter), by electrocautery (electrified wire at the tip of the cutting device), or other understood device/technique.

Various implementations of the catheter 20 further includes an optional pressure transducer (not shown) configured to detect pressure so as to allow the user to determine whether the catheter 20 is disposed in the aorta or elsewhere by measuring the transduced waveforms, as would be readily appreciated.

Various implementations of the catheter 20 include an end-to-end lumen shown for example in FIGS. 1, 4, and 6-8 extending the length of the catheter 20 to the distal end 22A of the balloon 26 or functional aspect 5.

The above applications can be in a native valve tissue or in a failed bioprosthetic valve. It is appreciated by those of skill in the art that they disclosed systems, devices and techniques can also be applied to other valves.

As would be appreciated, each of the individual sinuses of Valsalva 2 of the aortic valve can be treated sequentially as clinically needed.

Although the disclosure has been described with references to various implementations, persons skilled in the art will recognize that changes may be made in form and detail without departing from the spirit and scope of this disclosure.

What is claimed is:

1. A catheter comprising:
a) an elongate shaft comprising proximal and distal ends and a lumen; and
b) a functional aspect configured to navigate to and ablate aortic valve leaflet tissue inside a subject sinus of Valsalva.

2. The catheter of claim 1, wherein the functional aspect is an inflatable balloon or expandable member.

3. The catheter of claim 1, wherein the functional aspect is configured for targeted delivery of ablation energy or modification therapy to aortic valve leaflets.

4. The catheter of claim 1, wherein the functional aspect is configured for directional or differential delivery of ablation energy via electrodes to a leaflet of the aortic valve inside the sinus of Valsalva.

5. The catheter of claim 1, wherein the functional aspect comprises an active surface facing the valve leaflet tissue and passive or inert surface facing the aortic root wall in native valves or the cage of the bioprosthesis.

6. The catheter of claim 1, wherein the functional aspect is circular, semilunar or oval shaped.

7. The catheter of claim 1, further comprising at least one electrode wherein disposed on a surface of the expandable member or embodied in the functional aspect.

8. The catheter of claim 1, wherein the functional aspect is configured to apply energy to a leaflet of the aortic valve inside the sinus of Valsalva, the energy selected from the group consisting of electrical, thermal, radiofrequency (RFA), laser, microwave, ultrasound, ultraviolet light, pulse field or cooling energy.

9. The catheter of claim 8, configured to control at least one of frequency, duration, temperature, and power of the energy application.

10. The catheter of claim 1, wherein the functional aspect is a pigtail-shaped extension comprising one or more electrodes.

11. The catheter of claim 1, wherein the functional aspect is a clamp comprising first and second arms configured to sandwich the valve leaflet and deliver focused ablation energy between the first and second arms.

12. The catheter of claim 11, wherein the energy delivered by the clamp can be used to coagulate grasped leaflet tissue, cut the grasped leaflet tissue, or a combination of both.

13. The catheter of claim 11, further comprising a cutting device disposed between the first and second arms.

14. A method for performing transcatheter aortic valve replacement comprising:
inserting a catheter into an artery to a target valve;
positioning a distal end of the catheter adjacent to a leaflet of the target valve; and
lacerating the leaflet with a functional aspect of the catheter.

15. The method of claim 14, wherein the functional aspect is a clamp.

16. The method of claim 14, wherein the functional aspect is an expandable member having an active surface for transferring energy for lacerating the leaflet.

17. The method of claim 16, wherein the expandable member is a balloon.

18. The method of claim 16, wherein the active surfaces comprises one or more electrodes.

19. The method of claim 14, wherein the functional aspect of the catheter is a balloon and further comprising expanding the balloon via a heatable fluid, wherein the heatable fluid lacerates the leaflet.

20. A method of valvular repair comprising:
placing a catheter at a target valve;
expanding an expandable member to contact a leaflet of the target valve, the expandable member at a distal end of the catheter; and
lacerating the leaflet via radiofrequency energy transfer to one or more electrodes on a surface of the expandable member.
